SEQUENCE LISTING (14 sequences)

SEQ ID NO: 1 - DNA, 20 bp, Artificial Sequence
  Primer F CB1 (Accession N XM_013992672.2)
ttccccacttcttttccgcc

SEQ ID NO: 2 - DNA, 18 bp, Artificial sequence
  Primer R CB1 (Accession N XM_013992672.2)
gggagtcccttcgcatcc

SEQ ID NO: 3 - DNA, 20 bp, Artificial Sequence
  Primer F CB2 (Accession N XM_021095530.1)
tttatagcctggcctcccct

SEQ ID NO: 4 - DNA, 20 bp, Artificial Sequence
  Primer R CB2 (Accession N XM_021095530.1)
ttttcccgtctgcctctgtc

SEQ ID NO: 5 - DNA, 20 bp, Artificial Sequence
  Primer F FAAH (Accession N XM_013999418.2)
tgccaccgtgcaagaaaatg

SEQ ID NO: 6 - DNA, 20 bp, Artificial Sequence
  Primer R FAAH (Accession N XM_013999418.2)
ccactgccctaacaacgact

SEQ ID NO: 7 - DNA, 20 bp, Artificial Sequence
  Primer F DGL- (Accession N XM_021082924.1)
gaaaccaaacacgcctccac

SEQ ID NO: 8 - DNA, 20 bp, Artificial Sequence
  Primer R DGL- (Accession N XM_021082924.1)
caacccagcagcaaaggaac

SEQ ID NO: 9 - DNA, 20 bp, Artificial Sequence
  Primer F DGL- (Accession N XM_021086077.1)
tttgtaatcccggaccacgg

SEQ ID NO: 10 - DNA, 20 bp, Artificial Sequence
  Primer R DGL- (Accession N XM_021086077.1)
gacctgccgaggaatacgga

SEQ ID NO: 11 - DNA, 20 bp, Artificial Sequence
  Primer F TRPV1 (Accession N XM_005669121.1)
tcaccaacaagaaggggctc

SEQ ID NO: 12 - DNA, 20 bp, Artificial Sequence
  Primer R TRPV1 (Accession N XM_005669121.1)
ggataggtgcctgcactcag

SEQ ID NO: 13 - DNA, 20 bp, Artificial Sequence
  Primer F OR1G1 (Accession N XM_013990010.1)
cttggtttgtgtgctctgcc

SEQ ID NO: 14 - DNA, 20 bp, Artificial Sequence
  Primer R OR1G1 (Accession N XM_013990010.1)
gaaaaggctttccgcttccc

(19) United States
(12) Patent Application Publication
(10) Pub. No.: US 2023/0233482 A1
(43) Pub. Date: Jul. 27, 2023

(54) COMPOSITIONS COMPRISING THYMOL FOR USE IN THE TREATMENT OF INFLAMMATORY OR FUNCTIONAL BOWEL DISORDERS BY MODULATING THE ENDOCANNABINOID SYSTEM

(71) Applicant: VETAGRO INTERNATIONAL S.R.L., Reggio nell'Emilia (RE) (IT)

(72) Inventors: Andrea PIVA, Reggio nell'Emilia (RE) (IT); Ester GRILLI, Reggio nell'Emilia (RE) (IT)

(21) Appl. No.: 17/921,094

(22) PCT Filed: May 5, 2021
(86) PCT No.: PCT/IB2021/053803

§ 371 Date: Oct. 24, 2022

(30) Foreign Application Priority Data
May 5, 2020 (IT) 102020000009922

(51) Int. Cl. A61K 31/05; A61K 47/12; A61K 47/14; A61K 45/06; A23K 20/111; A23K 20/158; A23K 50/30; A23K 50/60

(52) U.S. Cl. CPC A61K 31/05; A61K 47/12; A61K 47/14; A61K 45/06; A23K 20/111; A23K 20/158; A23K 50/30; A23K 50/60

(57) ABSTRACT

The present invention relates to compositions comprising thymol for use in the preventive and/or curative treatment of inflammatory or functional diseases of the intestinal tract and related symptoms in human subjects, or monogastric animals, or poultry or fish by modulating the receptors and/or enzymes of the endocannabinoid system.

The present invention relates to compositions comprising thymol for use in the preventive and/or curative treatment of inflammatory or functional diseases of the intestinal tract and related symptoms in human subjects, or monogastric animals, or poultry or fish by modulating the receptors and/or enzymes of the endocannabinoid system.

Inflammatory or functional diseases or symptoms of the intestinal tract are widely common both in humans and in monogastric animals, negatively affecting their development—in particular in human or animal subjects in the growth phase—their quality of life and exposing them to the risk of developing further diseases due to the weakening of the immune system.

Among these, of particular importance are chronic inflammatory bowel diseases (IBDs), a group of nosological entities characterised by the presence of chronic phlogosis in the absence of infectious aetiology (i.e. not caused by bacteria, viruses or mycetes). The two most important diseases of the group are Crohn's disease Crohn and ulcerative rectocolitis IBD is a multifactorial disease, driven in part by an exaggerated immune response at the gut microbiota level that causes defects in the epithelial barrier function. In particular, the loss of integrity of the intestinal epithelium plays a key pathogenic role in IBD.

Most animals with IBD have a history of recurrent or chronic vomiting and/or diarrhoea. Often, a significant weight loss may be observed in animals affected by IBD, with all the entailed consequences.

Another gastrointestinal disease strongly common in humans and even in the monogastric animal population is irritable bowel syndrome (in short, IBS). Irritable bowel syndrome belongs to the group of functional gastrointestinal disorders (FGIDs), a diagnostic category that can be defined on the basis of the symptom presentation alone and characterised by the absence of an evident pathogenetic substrate (i.e. absence of bacteria, viruses or mycetes), in which abdominal discomfort or pain is associated with modifications of the gut microbiota. Depending on the characteristics of the faeces, four groups of the disease are distinguished: IBS with predominant constipation (constipated bowel), IBS with predominant diarrhoea (diarrhoeic bowel), IBS with alternating constipation and diarrhoea, unclassified IBS.

The drugs or compounds available to date for the treatment of inflammatory or functional diseases or symptoms of the intestinal tract in humans or monogastric animals often do not allow a complete and/or lasting resolution of the disease and the symptoms thereof. Furthermore, the drugs and treatments available on the market are not free of unwanted side effects which therefore sometimes limit their use as well.

Therefore, the interest on the part of the operators in the industry in being able to have a treatment that allows to overcome the limits and drawbacks still present in the known treatments and that is able to provide a rapid, effective and durable response against inflammatory or functional diseases or symptoms of the intestinal tract in humans, animals, birds, chickens and fish remains high.

The technical problem addressed and solved by the present invention lies in providing a composition and effective treatment for the treatment of intestinal disorders (diseases or symptoms) both of inflammatory and functional nature, mainly in the absence of bacterial, viral or fungal aetiology, in human subjects, in monogastric animals, preferably monogastric mammals, such as for example pigs or domestic animals, in poultry, preferably birds and chickens, and in fish, particularly also regarding the maintenance of a health condition and well-being of the intestine and/or support in the weaning (or initial growth) phase.

Following an intense research and development activity the Applicant addresses and solves the aforementioned technical problem by providing innovative mixtures, compositions (in short, mixtures and compositions of the invention) and treatments, having the characteristics as defined in the attached claims.

The present invention is also better disclosed with the aid of the following figures, provided solely by way of example and therefore not limiting the scope of protection thereof.

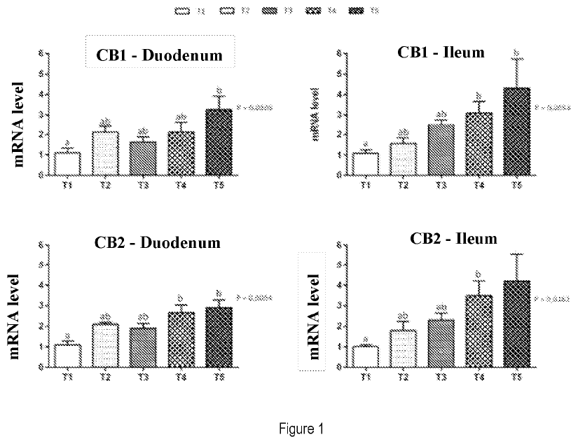

FIG. 1 reports the gene expression data for cannabinoid receptors in the duodenal and ileal mucosa at day 14 (d14) in pigs.

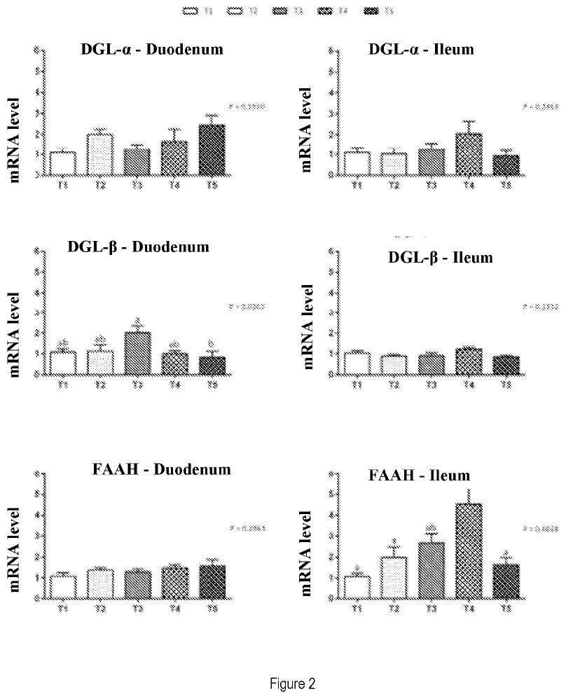

FIG. 2 reports the gene expression data for ECS enzymes in the duodenal and ileal mucosa at day 14 (d14) in pigs.

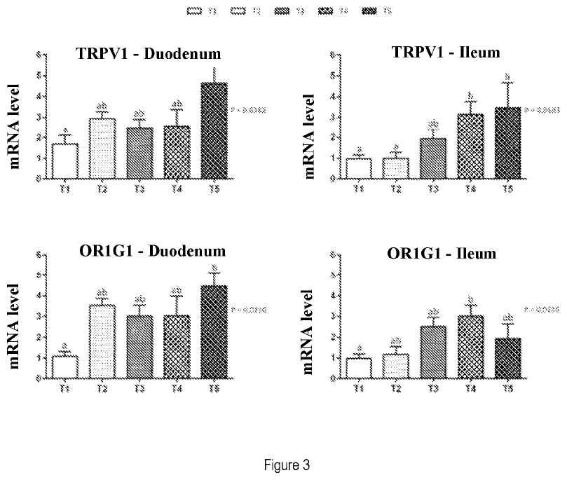

FIG. 3 reports the gene expression data for gut chemosensing in the duodenal and ileal mucosa at day 14 (d14) in pigs.

### DETAILED DESCRIPTION OF THE INVENTION

Forming an object of the present invention is a composition for use (in short, composition of the invention) in a method for the preventive and/or curative treatment of an inflammatory and/or functional bowel disease or of a related symptom by modulating (or modulation or enhancement of gene expression) of the receptors and/or enzymes of the endocannabinoid system and/or of the gut chemosensing system in a human being, or in a monogastric animal, or in a bird, or in a chicken, or in a fish, wherein said composition comprises a mixture M (in short, mixture M of the invention) comprising or, alternatively, consisting of thymol, and optionally, the composition further comprises at least one acceptable pharmaceutical or food grade additive and/or excipient.

The inflammatory and/or functional intestinal disease, or related symptom, is selected from intestinal diseases which do not mainly have microbial aetiology (bacterial, viral or fungal). Advantageously, the compositions of the present invention are validly used in the treatment of said diseases.

Advantageously, the compositions subject of the present invention show interesting therapeutic properties for the preventive and/or curative treatment of inflammatory or functional diseases of the intestinal tract thanks to the ability of thymol to modulate the intestinal gene expression of receptors of the endocannabinoid system (ECS), such as cannabinoid-1 (CB1) and cannabinoid-2 (CB2), and/or the enzymes of the endocannabinoid system (ECS), such as for example the fatty acid amide hydrolase (FAAH) involved in the degradation of the endocannabinoid molecule anandamide (AEA), and, in addition, to modulate one or more markers of the gut chemosensing system, such as for example transient receptor potential vanilloid-1 (TRPV1) and/or olfactory receptor 1G1 (OR1G1).

Advantageously, the compositions developed by the Applicant can be formulated and prepared so as to allow a gradual and targeted release into the intestine as a function of the species treated.

Forming an object of the present invention is a composition comprising, besides thymol, a controlled release lipid matrix embedding thymol and the other possible active components present in the composition of the invention. Said controlled release lipid matrix allows a gastroprotection (a protection from the acidic pH of the stomach), a controlled intestinal release of thymol and of the other active components, possibly present in the composition of the invention, preferably in a time comprised from 2 hours to 24 hours, preferably from 4 hours to 8 hours.

Activation of the endocannabinoid system (ECS) (receptors, signalling molecules and enzymes involved in ligand biosynthesis and degradation) is believed to be a valid approach in the control and in the regulation of inflammation and the functioning of the gastrointestinal system.

Cannabinoid receptors, whose main representatives are CB1 and CB2, are present in most of the organism including also in cells located in the intestine and digestive system. It has therefore been suggested that ECS regulates important physiological processes, including immune response, metabolism, digestive motility and appetite, and that it contributes to the maintenance of homeostasis, the sensitive internal balance of the organism. As a result, an irregular functioning of the endocannabinoid system, in particular modulation of the gene expression of receptors (CB1 and CB2) and enzymes, plays a decisive role in inflammatory and functional intestinal diseases.

Furthermore, it has been suggested that the “gut chemosensing system” may have therapeutic applications in the treatment of intestinal inflammatory or functional diseases due to the intestinal presence of a large variety of receptors, such as for example TRPV1 (transient receptor potential vanilloid 1) and OR1G1 (olfactory receptor 1G1).

The compositions or mixtures of the present invention have no significant side effects and they can be administered both to adult human subjects and animals, to human subjects and animals in the growth phase and to pregnant female animals.

Lastly, the compositions or mixtures of the present invention are easy to prepare and cost-effective.

When the composition of the invention does not include additives and/or excipients, the composition of the invention is identical to the mixture M of the invention.

Said treatment method of the present invention provides for the administration of a therapeutically effective amount of the composition or mixture M of the invention to the human subject, monogastric animal, poultry or fish.

In the context of the present invention, the expression “monogastric animals” is intended to include both mammalian and non-mammalian monogastric animals.

Furthermore, the composition or mixture M of the present invention comprising thymol is for use in maintaining gut health and/or for support in the weaning (or initial growth) phase of said human subjects or monogastric animals, preferably monogastric mammals.

Preferably, said monogastric animal is a monogastric mammal, in particular a monogastric animal or monogastric mammal in the weaning phase. Furthermore, in the context of the present invention poultry, such as for example birds and chickens, and fish are also included.

Said monogastric mammals, adult or in the weaning phase, which can be treated by means of the compositions or mixtures M of the invention, may be selected from: dogs, cats, monkeys, pigs, equines, such as horses and donkeys, rabbits before weaning, rodents, such as hamsters, cavies, mice, gerbils, chinchillas, degus, squirrels, guinea pigs and rats; weasels, ferrets and ermines.

Said non-mammalian monogastric animals, adult or in initial growth phase, that can be treated using the compositions or mixtures M of the invention, can be selected from: animals of the poultry species (class Ayes, preferably order Galliformes), such as chicken or other poultry, turkey, guinea fowl, pheasant, peacock, partridge, quail, dove, turtle dove, goose, common duck and Muscovy duck, or, alternatively, an animal belonging to the aquatic species, such as fish and crustaceans.

The diseases or symptoms that can be preventively and/or curatively treated using the compositions or mixtures M of the invention, according to any one of the described embodiments, are advantageously selected from: chronic irritable bowel disease (IBD), Crohn's syndrome or disease, ulcerative colitis, indeterminate colitis, microscopic colitis, collagenous colitis, lymphocytic colitis, ischemic colitis, diversion colitis, pouchitis, irritable bowel syndrome (IBS), diarrhoeic IBS, constipation IBS, alternating IBS, unclassified IBS, dyspepsia, nausea, vomiting, constipation, diarrhoea, abdominal bloating, tympanites and physical fatigue.

The compositions or mixtures M of the present invention, according to any of the described embodiments, may be for use as adjuvants of further therapeutic approaches (e.g. drugs) in the treatment of the diseases indicated in the present invention.

Besides thymol, the mixture M contained in the composition of the invention may further comprise a further first active ingredient, for example deriving from a phytocompound (botanicals), selected from group (I) comprising or, alternatively, consisting of carvacrol, eugenol, capsaicin, turmeric, vanillin, cinnamaldehyde, diallyl disulfide, camphor, limonene, rosmarinic acid, p-cymene, γ-terpinene, α-pinene, α-thujone, 1,8-cineole, verbascoside, tannins, menthol, linalool (linalool C10H18O (CAS No. 78-70-6), terpineol (terpineol C10H18O (CAS No. 98-55-5), anthocyanins (anthocyanin), catechins, alpha-zingiberene (C15H24 CAS No. 495-60-3) and mixtures thereof.

In an embodiment, the composition of the invention may comprise thymol and carvacrol, or thymol and vanillin, or thymol and menthol, or thymol and linalool (linalool C10H18O (CAS No. 78-70-6), or thymol and terpineol (terpineol C10H18O (CAS No. 98-55-5), or thymol and anthocyanins (anthocyanin), or thymol and catechins, or thymol and alpha-zingiberene (C15H24 CAS No. 495-60-3) and mixtures thereof; or thymol, carvacrol and capsaicin, or thymol, carvacrol and cinnamaldehyde, or thymol, eugenol and verbascoside.

Besides thymol and, optionally, said further first active ingredient (botanicals) selected from group (I), the mixture M, contained in the composition of the invention may further comprise an organic acid or a salt thereof with an alkaline or alkaline earth metal cation, wherein said organic acid is selected from group (II) comprising or, alternatively, consisting of lactic acid, malic acid, benzoic acid, fumaric acid, sorbic acid, citric acid, octanoic acid, heptanoic acid, butyric acid, dodecanoic acid and mixtures thereof; preferably butyric acid, benzoic acid, dodecanoic acid and mixtures thereof. For example, the composition of the invention may comprise thymol and sorbic acid; thymol, sorbic acid and citric acid; thymol and benzoic acid; thymol, sorbic acid, citric acid and benzoic acid; thymol, carvacrol and sorbic acid; thymol, carvacrol, sorbic acid and citric acid; thymol, carvacrol, cinnamaldehyde and sorbic acid, or thymol, carvacrol, cinnamaldehyde, sorbic acid and citric acid, thymol and butyric acid, thymol, citric acid and dodecanoic acid; preferably thymol and butyric acid, or thymol and benzoic acid, or thymol and dodecanoic acid. In the context of the present invention both thymol and said further first active ingredient (botanicals) selected from group (I) can be in the form of essential oils, or oleoresins, or plant extracts, the significant aspect lies in the fact that said essential oils or oleoresins or plant extracts contain the molecules described above (thymol and botanicals of group (I)),

In the mixture M, contained in the composition of the present invention, the molar ratio between A and B, wherein: A is thymol and, optionally, at least one or more of said further first active ingredients (botanicals) selected from group (I), while B is at least one or more organic acids or salts thereof selected from group (II), is comprised in the range from 1:500 to 500:1, preferably from 1:300 to 300:1, more preferably from 1:100 to 100:1 or from 1:50 to 50:1 or from 1:10 to 10:1.

Besides thymol and, optionally, said further first active ingredient selected from group (I) and/or said organic acid or a salt thereof selected from group (II), according to any of the described embodiments, the mixture M contained in the composition of the invention may further comprise at least one further second active ingredient selected from group (III) consisting of:
bacterial strains or probiotic bacterial strains belonging to the genus Lactobacillus, Streptococcus, Leuconostoc, Bifidobacterium, Pediococcus, Enterococcus, Saccaromyces;
prebiotics, such as for example inulin, lactulose, lactitol, mannooligosaccharides, fructooligosaccharides and galactooligosaccharides, tributyrin, monobutyrin (monobutyrin C7H14O4 CAS No. 557-25-5), monolaurin (monolaurin C15H30O4 CAS No. 27215-38-9) and mixtures thereof;
salts of metals, such as zinc and copper; and mixtures thereof.

Furthermore, the composition of the present invention may comprise a controlled release lipid matrix embedding or incorporating said mixture M which comprises thymol and, optionally, further ingredients selected from group (I) and/or (II) and/or (III), according to any one of the described embodiments. Said controlled release lipid matrix comprises or, alternatively, consists of at least one saturated or unsaturated, free or esterified fatty acid, having a number of carbon atoms comprised in the range from C10-C30, preferably from C14-C24 or C16-C22, and/or at least one triglyceride having saturated or unsaturated fatty acid chains having a number of carbon atoms comprised in the range from C6-C30, preferably from C14-C24 or C17-C21, and/or at least one wax having a number of carbon atoms comprised in the range from C16-C36, preferably from C24-C36 or C26-032; wherein said lipid matrix allows a gastroprotection from the acidic pH of the stomach and ensures an intestinal controlled release of thymol and of the further components possibly present in the mixture M, as a function of the species to be treated and of the type of lipid matrix used. The controlled release may occur over a time comprised in the range from 2 hours to 24 hours, preferably from 4 hours to 8 hours.

The matrix M and the composition containing it are prepared using techniques and equipment known to the person skilled in the art. In particular, the process for preparing the matrix M provides for mixing, in a mixer or in a container provided with stirring or mixing and heating means, the thymol and, optionally, one or more of the further ingredients selected from group (I) and/or (II) and/or (III), according to any of the described embodiments, together with the lipid matrix so that all the compounds and ingredients are embedded together in the matrix as a whole.

“Triglycerides” (or triacylglycerols) are neutral esters of glycerol in which chains of three long chain fatty acids are present instead of the hydrogen atoms of the hydroxyl groups. The length of the fatty acid chains in the common triglycerides structures may be from 5 to 28 carbon atoms, but 17 and 19 are more common.

The term “fatty acids” (in short FAs) is mainly but not exclusively used to indicate long-chain aliphatic monocarboxylic acids (number of carbon atoms comprised in the range C10-C30) with an even number of carbon atoms, without branching and acyclic (i.e., consisting of molecules that do not have ring-like closed chains). Fatty acids can be saturated (if their molecule has single C—C bonds only) or unsaturated (if they have double C═C bonds).

The term “waxes” is used to indicate to long-chain fatty acid esters with high molecular weight monohydric alcohols. Waxes may be of plant origin or animal origin (beeswax). Beeswax consists of various compounds, including: hydrocarbons 14%, monoesters 35%, diesters 14%, triesters 3%, hydroxy monoesters 4%, hydroxy polyesters 8%, acid esters 1%, acidic polyesters 2%, free acids 12%, free alcohols 1%, not identified 6%, The main components of beeswax are palmitates, palmitic acid, hydroxypalmitates and oleate esters formed by long chains (30-32 carbon atoms) of aliphatic alcohols, with a 6:1 ratio between the two main components triacontanyl palmitate (myricyl palmitate) CH₃(CH₂)₂₉O—CO—(CH₂)14 CH₃ and cerotic acid CH₃(CH₂)₂₄COOH. Beeswax has a melting comprised between 62° C. and 64° C. Density at 15° C. ranges between 0.958 g/cm³ and 0.970 g/cm³. Beeswax can be classified into two broad categories: European type and Eastern type. The saponification number is 3-5 for European type and 8-9 for Eastern type.

Said triglyceride comprised in said controlled release lipid matrix may be a hydrogenated or non-hydrogenated triglyceride of plant or animal origin, preferably hydrogenated triglyceride of plant and/or animal origin, more preferably a hydrogenated triglyceride of plant origin.

Said fatty acid comprised in the controlled release lipid matrix may be a hydrogenated or non-hydrogenated fatty acid, or an ester thereof of plant and/or animal origin, preferably a hydrogenated fatty acid of plant and/or animal origin, more preferably a hydrogenated fatty acid of plant origin.

Said waxes comprised in the controlled release lipid matrix may be of plant and/or animal origin; preferably beeswax.

Examples of said fatty acid, triglyceride or wax of plant origin, even in the hydrogenated form, are: palm oil, sunflower oil, corn oil, rapeseed oil, peanut oil, soybean oil, olive oil, beeswax, and mixtures thereof.

Examples of triglycerides of animal origin, also in the hydrogenated form, are selected from: chicken fat, hydrogenated chicken fat, bovine tallow and pork lard.

The composition of the invention, may comprise the mixture M, comprising thymol and, optionally, at least one further ingredient selected from group (I), (II) and/or (III) according to any one of the described embodiments, in a % by weight comprised in the range from 1% to 80%, preferably from 10% to 50%, more preferably from 15% to 45%, and said controlled release matrix, according to any one of the embodiments of the invention, in a % by weight comprised in the range from 10% to 80%; preferably from 40% to 60%, more preferably from 45% to 55%, wherein said % are with respect to the total weight of the composition.

The composition of the invention, according to any of the described embodiments, may be a feed or a feed additive.

Alternatively, said composition of the invention, according to any one of the described embodiments, may be a pharmaceutical composition, a medical device composition, a dietary supplement, a food (or novel food or food for special medical purposes), a composition for a dietary supplement, both for human subjects and for animals (veterinary products).

Advantageously, the mixtures or compositions of the present invention, according to any of the described embodiments, are formulated for oral use.

The composition of the present invention may be formulated for oral use in solid form, for example, granules, flakes, powder, soluble powder or granules, tablets, capsules; or, alternatively, in liquid form, for example, selected from: solutions, suspensions, emulsions, liquid which can be dispensed in the form of sprays, syrups; or, alternatively, in semi-liquid form, for example, selected from: soft-gels, gels.

Preferably the composition of the invention is for oral use in solid form, for example granules, powder or flakes.

Advantageously, in order to be effective in the treatment methods described in the present invention, the compositions of the invention are administered to an animal in need in a daily dose comprising thymol in an amount (mg/kg of feed) comprised in the range from 5 mg/kg to 5000 mg/kg, preferably from 10 mg/kg to 2000 mg/kg, more preferably from 15 mg/Kg to 1000 mg/Kg.

The aforementioned daily doses may be administered to the subject in need in a single dose (one dose) or in repeated doses, for example two, three or four daily doses.

Lastly, forming an object of the present invention is the use of said feed or a feed additive comprising the composition or mixture M of the present invention, according to one of the described embodiments, for weaning or supporting the initial growth of a monogastric animal; preferably a monogastric mammal, more preferably pigs.

In order to achieve the object of the present invention, the components (or active components) of the mixture M of the invention, such as thymol, components of group (I), (II) and/or (III), may be administered to an animal in need also separately and sequentially, and in any order; for example, in a close sequence over time (from about 0 minutes to 30 minutes) or in a non-close sequence over time (from 1 hour to about 4 or 6 or 8 or 12 hours), and administered at the same or different frequency. When said active components of the mixture M of the invention are administered in a single composition, said single composition corresponds to the composition of the present invention.

Unless specified otherwise, the expression composition or mixture or other comprising a component at an amount “comprised in a range from x to y” is used to indicate that said component can be present in the composition or other at all the amounts present in said range, even though not specified, extremes of the range comprised.

Unless specified otherwise, the indication that a composition “comprises” one or more components or substances means that other components or substances can be present besides the one, or the ones, indicated specifically.

In the context of the present invention, the expression “treatment method” is used to indicate an intervention on a subject in need, comprising the administration of the bacterial strain or of a composition of the invention with the aim of eliminating, reducing/decreasing or preventing a disease or ailment and the symptoms or disorders thereof.

The term “therapeutically effective amount” refers to the amount of active compound and/or bacterial strain that elicits the biological or medicinal response in a tissue, system, mammal, or human being that is sought and defined by an individual, researcher, veterinarian, physician, or other clinician or health worker.

In the context of the present invention, the term “medical device” is used in the meaning according to the Legislative Decree no 46 dated 24 Feb. 1997, or in accordance with the new Medical device regulation (EU) 2017/745 (MDR).

In the context of the present invention, the term “novel food” is used in the meaning according to Regulation EC 258 dated 1997.

Forming an object of the present invention is a process for the preparation of the compositions described above, preferably from FR1 to FR10 and from E1 to E10, which provides for a step in which oil-in-water emulsions and, separately, water-in-oil emulsions are prepared, said emulsions containing the ingredients of group (I) and/or group (II) and/or group (III) which constitute the mixture to obtain a composition in solid form.

Embodiments FRn of the present invention are reported below.

FR1. A composition for use in a method for preventive and/or curative treatment of, an inflammatory and/or functional bowel disease or of a related symptom by modulating receptors and/or enzymes of the endocannabinoid system, in a human being, or in a monogastric animal, or in birds, or in fish,

wherein said composition comprises:
a mixture M comprising or, alternatively, consisting of thymol;
   and, optionally,
at least one acceptable pharmaceutical or food grade additive and/or excipient.

FR2. The composition for use according to FR1, wherein said composition is for use in a monogastric mammal, such as in pigs; preferably a monogastric animal or monogastric mammal in the weaning phase, like in weaning pigs.

FR3. The composition for use according to any one of FRs 1-2, wherein said disease or symptom is selected from: chronic irritable bowel disease (IBD), Crohn's syndrome or disease, ulcerative colitis, indeterminate colitis, microscopic colitis, collagenous colitis, lymphocytic colitis, ischemic colitis, diversion colitis, pouchitis, irritable bowel syndrome (IBS), diarrhoeic IBS, constipation IBS, alternating IBS, unclassified IBS, dyspepsia, nausea, vomiting, constipation, diarrhoea, abdominal bloating, tympanites and physical fatigue.

FR4. The composition for use according to any one of FRs 1-3, wherein said composition further comprises a controlled release lipid matrix embedding or incorporating said mixture M comprising or, alternatively, consisting of thymol wherein said controlled release lipid matrix comprises or, alternatively, consists of at least one saturated or unsaturated, free or esterified fatty acid having a number of carbon atoms comprised in the range from C10-C30, and/or at least one triglyceride having saturated or unsaturated fatty acid chains, having a number of carbon atoms comprised in the range from C6-C30 and/or at least one wax having a number of carbon atoms comprised in the range from C16-036; wherein said lipid matrix allows a gastroprotection and/or a controlled intestinal release of the mixture M, preferably of the thymol contained therein.

FR5. The composition for use according to any one of FRs 1-4, wherein said controlled release lipid matrix comprises or, alternatively, consists of at least one hydrogenated fatty acid of plant origin having a number of carbon atoms comprised in the range from C14-C24 and/or at least one hydrogenated triglyceride of plant origin having a number of carbon atoms comprised in the range from C14-C24 and/or at least one wax of animal origin having a number of carbon atoms comprised in the range from C24-036; preferably, wherein said fatty acid, triglyceride or wax is selected from: palm oil, sunflower oil, corn oil, rapeseed oil, peanut oil, soybean oil, olive oil, beeswax, and mixtures thereof.

FR6. The composition for use according to any one of FRs 1-5, wherein said mixture M further comprises at least one further first active ingredient, preferably deriving from a phytocompound (botanicals), selected from group (I) comprising or, alternatively, consisting of carvacrol, eugenol, capsaicin, turmeric, vanillin, cinnamaldehyde, diallyl disulfide, camphor, limonene, rosmarinic acid, p-cymene, γ-terpinene, α-pinene, α-thujone, 1,8-cineole, verbascoside, tannins and mixtures thereof.

FR7. The composition for use according to any one of FRs 1-6, wherein said composition comprises thymol and carvacrol, or thymol and vanillin, or thymol, carvacrol and capsaicin, or thymol, carvacrol and cinnamaldehyde, or thymol, eugenol and verbascoside.

FR8. The composition for use according to any one of FRs 1-7, wherein the mixture M contained in said composition comprises, besides thymol and, optionally, said further first active ingredient (botanicals) selected from group (I), an organic acid or a salt thereof with an alkaline or alkaline earth metal cation, wherein said organic acid is selected from group (II) comprising or, alternatively, consisting of lactic acid, malic acid, benzoic acid, fumaric acid, sorbic acid, citric acid, octanoic acid, heptanoic acid, butyric acid, dodecanoic acid and mixture thereof; preferably it may comprise thymol and sorbic acid, or thymol, sorbic acid and citric acid, or thymol and benzoic acid, or thymol, sorbic acid, citric acid and benzoic acid, or thymol, carvacrol and sorbic acid, or thymol, carvacrol, sorbic acid and citric acid, or thymol, carvacrol, cinnamaldehyde and sorbic acid, or thymol, carvacrol, cinnamaldehyde, sorbic acid and citric acid, or thymol and butyric acid, or thymol, citric acid and dodecanoic acid.

FR9. The composition for use according to any one of FRs 1-8, wherein said mixture M further comprises at least one further second active ingredient selected from group (III) consisting of:
bacterial strains or probiotic bacterial strains belonging to the genus Lactobacillus, Bifidobacterium, Streptococcus, Leuconostoc, Pediococcus, Enterococcus, Saccaromyces; and/or
prebiotics, such as for example inulin, lactulose, lactitol, mannan-oligosaccharides, fructigosaccharides and galacto-oligosaccharides, tributyrin; and/or
salts of metals, such as zinc and copper; and mixtures thereof.

FR10. The composition for use according to any one of FRs 1-9, wherein said composition is a feed or feed additive for monogastric animals and/or monogastric animals in the weaning or initial growth phase; preferably for monogastric mammals, more preferably for pigs.

E1. A composition for use in a method for preventive and/or curative treatment of, an inflammatory and/or functional bowel disease or of a related symptom by modulating receptors and/or enzymes of the endocannabinoid system, in a human being, or in a monogastric animal, or in birds, or in fish,

wherein said composition comprises:
a mixture M comprising or, alternatively, consisting of thymol;
   wherein said composition further comprises a controlled release lipid matrix embedding or incorporating said mixture M comprising or, alternatively, consisting of thymol wherein said controlled release lipid matrix comprises or, alternatively, consists of at least one saturated or unsaturated, free or esterified fatty acid having a number of carbon atoms comprised in the range from C10-C30, and/or at least one triglyceride having saturated or unsaturated fatty acid chains, having a number of carbon atoms comprised in the range from C6-C30 and/or at least one wax having a number of carbon atoms comprised in the range from C16-C36;
   wherein said lipid matrix allows a gastroprotection and/or a controlled release of the mixture M, preferably of the thymol contained therein, into the bowel;
   and, optionally,
at least one acceptable pharmaceutical or food grade additive and/or excipient.

E2. The composition for use according to E1, wherein said composition is for use in a monogastric mammal, such as in pigs; preferably a monogastric animal or monogastric mammal in the weaning phase, like in weaning pigs.

E3. The composition for use according to any one of E1-E2, wherein said disease or symptom is selected from: chronic irritable bowel disease (IBD), Crohn's syndrome or disease, ulcerative colitis, indeterminate colitis, microscopic colitis, collagenous colitis, lymphocytic colitis, ischemic colitis, diversion colitis, pouchitis, irritable bowel syndrome (IBS), diarrhoeic IBS, constipation IBS, alternating IBS, unclassified IBS, dyspepsia, nausea, vomiting, constipation, diarrhoea, abdominal bloating, tympanites and physical fatigue.

E4. The composition for use according to any one of E1-E3, wherein said controlled release lipid matrix comprises or, alternatively, consists of at least one hydrogenated fatty acid of plant origin having a number of carbon atoms comprised in the range from C14-C24 and/or at least one hydrogenated triglyceride of plant origin having a number of carbon atoms comprised in the range from C14-C24 and/or at least one wax of animal origin having a number of carbon atoms comprised in the range from C24-C36; preferably, wherein said fatty acid, triglyceride or wax is selected from: palm oil, sunflower oil, corn oil, rapeseed oil, peanut oil, soybean oil, olive oil, beeswax, and mixtures thereof.

E5. The composition for use according to any one of E1-E4, wherein said mixture M further comprises at least one further first active ingredient, preferably deriving from a phytocompound (botanicals), selected from group (I) comprising or, alternatively, consisting of carvacrol, eugenol, capsaicin, turmeric, vanillin, cinnamaldehyde, diallyl disulfide, camphor, limonene, rosmarinic acid, p-cymene, γ-terpinene, α-pinene, α-thujone, 1,8-cineole, verbascoside, tannins, menthol, linalool (linalool C10H18O (CAS No. 78-70-6), terpineol (terpineol C10H18O (CAS No. 98-55-5), anthocyanins (anthocyanin), catechins, alpha-zingiberene (C15H24 CAS No. 495-60-3) and mixtures thereof.

E6. The composition for use according to any one of E1-E5, wherein said composition comprises thymol and carvacrol, or thymol and vanillin, or thymol and menthol, or thymol and linalool (linalool C10H18O (CAS No. 78-70-6), or thymol and terpineol (terpineol C10H18O (CAS No. 98-55-5), or thymol and anthocyanins (anthocyanin), or thymol and catechins, or thymol and alpha-zingiberene (C15H24 CAS No. 495-60-3) and mixtures thereof; or thymol, carvacrol and capsaicin, or thymol, carvacrol and cinnamaldehyde, or thymol, eugenol and verbascoside.

E7. The composition for use according to any one of E1-E6, wherein the mixture M contained in said composition comprises, besides thymol and, optionally, said further first active ingredient (botanicals) selected from group (I), an organic acid or a salt thereof with an alkaline or alkaline earth metal cation, wherein said organic acid is selected from group (II) comprising or, alternatively, consisting of lactic acid, malic acid, benzoic acid, fumaric acid, sorbic acid, citric acid, octanoic acid, heptanoic acid, butyric acid, dodecanoic acid and mixture thereof; preferably it may comprise thymol and sorbic acid, or thymol, sorbic acid and citric acid, or thymol and benzoic acid, or thymol, sorbic acid, citric acid and benzoic acid, or thymol, carvacrol and sorbic acid, or thymol, carvacrol, sorbic acid and citric acid, or thymol, carvacrol, cinnamaldehyde and sorbic acid, or thymol, carvacrol, cinnamaldehyde, sorbic acid and citric acid, or thymol and butyric acid, or thymol, citric acid and dodecanoic acid.

E8. The composition for use according to any one of E1-E7, wherein said mixture M further comprises at least one further second active ingredient selected from group (III) consisting of:
bacterial strains or probiotic bacterial strains belonging to the genus Lactobacillus, Bifidobacterium, Streptococcus, Leuconostoc, Pediococcus, Enterococcus, Saccaromyces; and/or
prebiotics, such as for example inulin, lactulose, lactitol, mannooligosaccharides, fructooligosaccharides and galactooligosaccharides, tributyrin; monobutyrin (monobutyrin C7H14O4 CAS No. 557-25-5), monolaurin (monolaurin C15H30O4 CAS No. 27215-38-9) and mixtures thereof and/or
salts of metals, such as zinc and copper; and mixtures thereof.

E9. The composition for use according to any one of E1-E8, wherein said composition is a feed or feed additive for monogastric animals and/or monogastric animals in the weaning or initial growth phase; preferably for monogastric mammals, more preferably for pigs.

E10. A process for the preparation of the compositions according to any one of E1 to E9, wherein said process provides for a step in which oil-in-water emulsions and, separately, water-in-oil emulsions are prepared, said emulsions containing the ingredients of group (I) and/or group (II) and/or group (III) which constitute the mixture to obtain a composition in solid form.

### Experimental Part

The purpose of this study was to study the presence of markers of the endocannabinoid system and of the pig gut chemosensing system and, secondly, to determine how thymol modulates these markers.

### Method

### Ethics Statement

The study was conducted at the facilities of the Research Centre for Animal Production and Environment (CER-O), which is Good Laboratory Practices-certified and operates according to the Procedure of Animal Protection and Welfare (directive No 86/609/EEC) Animals used in the study were raised and treated according to European Union directive 2010/63/EU. The study was authorized by the Italian Ministry of Health according to art. 31 of the Italian legislative decree no 26/2014 and on Commission recommendation 2007/526/EC, which covers the housing and care of animals used for experimental and other scientific purposes (authorization no 341/2017-PR issued May 3, 2017). Animals were obtained from a breeding farm in Cascina Mandellina, Bergamo, Italy.

### Animals and Diets

One hundred and fifty piglets (Duroc—Large White) weaned at 28 days of age and with a body weight (BW) of 7.71±1.00 kg were divided into 40 pens (4 piglets per pen, castrated males and females were placed in separate pens) and randomly assigned to one of the following experimental groups (n=32): control group fed with the basal diet (T1), a group fed with the basal diet supplemented with 25.5 mg of thymol/kg feed (T2), a group fed with the basal diet supplemented with 51 mg of thymol/kg feed (T3), a group fed with the basal diet supplemented with 153 mg of thymol/kg feed (T4) and a group fed with the basal diet supplemented with 510 mg of thymol/kg feed (T5). Thymol was provided in a form embedded (microencapsulated) in a lipid matrix (Vetagro SpA, Reggio Emilia, Italy). Concentrations of thymol were selected to meet or exceed the upper limit of inclusion in food and feed established by the European Agency for the Evaluation of Medicinal Products and for the feed. The basal feed was formulated to meet or exceed the nutritional requirements of pigs according to the National Research Council, and feed and water were provided ad libitum (the composition of the basal diet is reported in Table 1). The health condition of the animals was monitored during the study. The piglets were individually weighed at the beginning (day 0) and end (day 14) of the study. Growth parameters, such as FI, ADFI, ADG and F:G (FI—feed intake; ADFI—Average daily feed intake; ADG—Average daily gain; F:G—Feed to gain ratio), were measured in the animals housed in each pen on day 14 (d14) of the experiment.

TABLE 1
Ingredient (% dry matter)

| | |
|---|---|
| Corn meal | 59.25 |
| Soybean meal, 44% | 21.90 |
| Sweet milk whey | 8.00 |
| Fishmeal (Herring 999) | 7.00 |
| Soybean oil | 1.98 |
| Calcium carbonate | 0.35 |
| Vitamin and mineral premix | 0.25 |
| L-lysine HCl | 0.54 |
| NaCl | 0.16 |
| L-threonine | 0.24 |
| DL-methionine | 0.26 |
| L-tryptophan | 0.08 |
| Total | 100 |

At the end of the study, 8 animals from each treatment group were sacrificed, samples were collected, and analysed. Duodenal and ileal mucosal scrapings were collected. The duodenum and ileum were longitudinally cut to expose the mucosa, washed with a phosphate-buffered saline solution to remove mucus and digesta, then scraped gently, packed, immediately frozen in liquid nitrogen and stored at −80° C. until the analyses of gene and protein expression.

### Gene Expression Analysis

Gene expression was analysed using the method reported by Herfel et al. [The Journal of Nutrition. 2011; 141:2139-45]. Duodenal and ileal scraping samples obtained on d14 of the study were disrupted by grinding them in liquid nitrogen with mortar and pestle, and then homogenised using a TissueLyser (Qiagen, Hilden, Germany). Total RNA was isolated using a NucleoSpin® RNA Kit (Macherey-Nagel, Düren, Germany) according to the manufacturer's instructions. Genomic DNA contamination was removed by treating the samples with the deoxyribonuclease supplied in the extraction kit (rDNase, RNase-free; Macherey-Nagel). The RNA yield and quality were determined spectrophotometrically by measuring the absorbance at 260 and 280 nm (A260 and A280 nm, respectively) (Microvolume Mode with SmartPath® Technology, Denovix). One microgram of RNA was reverse transcribed using the iScript cDNA Synthesis Kit (Bio-Rad Laboratories Inc., Hercules, Calif., USA) according to the manufacturer's instructions. Real-time PCR was performed using an iCycler Thermal Cycler system and SybrGreen Supermix (Bio-Rad Laboratories Inc.). The thermocycling protocol provided for an initial denaturation step for 1 min and 30 seconds at 95° C., followed by 40 cycles of denaturation at 95° C. for 15 seconds and 30 seconds of annealing and extension at 60° C. After amplification, a melting curve analysis was performed for all the samples, with slow heating from 55° C. to 95° C. at a rate of 0.5° C./s to validate the absence of non-specific products. Gene expression was normalised to a housekeeping gene (HK) encoding portions of porcine ribosomal subunit 60 S, in particular ribosomal protein L35 (RPL35). The average threshold cycle (CT) was determined for each gene of interest, and the geometric average was calculated for HK by assuming that CT is the number of cycles required to reach a fixed arbitrary threshold. Delta CT was calculated, then a modification of the 2^{-ΔΔCT} method [Livak K J, et al., Methods 2001; 25:402-8] was used to analyse the relative expression (fold changes), which was calculated relative to the control group. The sequences, accession numbers in the EMBL database/GenBank, expected product lengths and references for porcine primers are reported in Table 2. Primer oligonucleotides for CB1, DGL-β and OR1G1 were designed using the Primer-BLAST tool (NCBI National Center for Biotechnology Information, www.ncbi.nlm.nih.gov). Primers were obtained from Life Technologies (Life Technologies Italia).

TABLE 2

| | Primer sequence (F and R) | | |
|---|---|---|---|
| Gene | 5′ → 3′ | bp | Accession No |
| CB1 | F: TTCCCCACTTCTTTTCCGCC | 208 | XM_013992672.2 |
| | R: GGGAGTCCCTTCGCATCC | | |
| CB2 | F: TTTATAGCCTGGCCTCCCCT | 240 | XM_021095530.1 |
| | R: TTTTCCCGTCTGCCTCTGTC | | |
| FAAH | F: TGCCACCGTGCAAGAAAATG | 234 | XM_013999418.2 |
| | R: CCACTGCCCTAACAACGACT | | |
| DGL-α | F: GAAACCAAACACGCCTCCAC | 211 | XM_021082924.1 |
| | R: CAACCCAGCAGCAAAGGAAC | | |
| DGL-β | F: TTTGTAATCCCGGACCACGG | 255 | XM_021086077.1 |
| | R: GACCTGCCGAGGAATACGGA | | |
| TRPV1 | F: TCACCAACAAGAAGGGGCTC | 116 | XM_005669121.1 |
| | R: GGATAGGTGCCTGCACTCAG | | |
| OR1G1 | F: CTTGGTTTGTGTGCTCTGCC | 96 | XM_013990010.1 |
| | R: GAAAAGGCTTTCCGCTTCCC | | |

bp: product length; F: forward; R: Reverse

### Statistical Analysis

Animals were blocked in a completely randomised design and data were analysed using GraphPad Prism® software (GraphPad Software, Inc., La Jolla, Calif., USA). Data were analysed using one-way ANOVA followed by Tukey's post hoc test to detect differences among treatments. The pen was the experimental unit for growth performance, whereas the pig was the experimental unit for gene expression. Differences were considered significant at P<0.05, and trends were defined at 0.05≤P<0.1.

### Results

### Growth Performance

The piglets maintained a good health conditions during the experiment and no mortality was recorded. During the experiment, differences in body weight (BW), feed intake (FI), average daily feed intake (ADFI), average daily gain (ADG), and feed to gain ratio (F:G) were not observed among the treatment groups (data not shown).

### Endocannabinoid System (ECS)

FIG. 1 summarises gene expression data for cannabinoid receptors in the duodenal and ileal mucosa at day 14 (d14). Cannabinoid receptor 1 and 2 mRNAs were detected in both the duodenal and ileal mucosa. The level of the CB1 mRNA was significantly increased in the duodenum of group T5 (P=0.0209) and in the ileum of groups T4 and T5 (P=0.0054) compared to the control group. Significantly increased levels of the CB2 mRNA were detected in both the duodenum and ileum of groups T4 and T5 compared to the control group (P=0.004 and P=0.0162 respectively). Data on gene expression for ECS enzymes are reported in FIG. 2. The presence of mRNA for all the enzymes tested was confirmed. Differences in FAAH mRNA levels were not observed in duodenum, while FAAH mRNA levels were significantly increased in the ileum of group T4 compared to the control group (P=0.0028).

### Gut Chemosensing System

The results for the gut chemosensing are reported in FIG. 3. As regards the gut chemosensing markers, both TRPV1 and OR1G1 (Olfactory receptor 1G1) mRNAs were detected in the duodenal and ileal mucosa. Furthermore, completion of 510 mg thymol/kg of feed increased the TRPV1 mRNA level in the duodenum (P=0.0382), while increased TRPV1 mRNA levels were observed in the ileum of groups T4 and T5 compared to the control group (P=0.0183). The OR1G1 mRNA was expressed at higher levels in the duodenum of animals fed with feed supplemented with 510 mg thymol/kg feed (T5) (P=0.0210) and in the ileum of animals fed with 153 mg thymol/kg feed (T4) (P=0.0235) compared to the control group.

### CONCLUSIONS

In conclusion, the data of the present study not only confirm the presence of markers of the endocannabinoid system (ECS) and of the gut chemosensing n the duodenal and ileal mucosa of the piglets, but it also demonstrates that thymol modulates the gene expression of these markers. Thymol increases the expression of the mRNAs encoding the CB1 and CB2 receptors both in the duodenum and ileum. Thymol also modulates mRNA levels of enzymes involved in biosynthesis and degradation of endocannabinoid molecules (e.g. FAAH). Furthermore, the upregulation of OR1G1 and TRPV1 (chemosensory receptors) performed by thymol in the intestine demonstrates a possible role of thymol as a feeding additive in the enhancement of the intestinal health of the animal, particularly in the weaning and growth phase.

1. A composition for use in a method for preventive and/or curative treatment of, an inflammatory and/or functional bowel disease or of a related symptom by modulating receptors and/or enzymes of the endocannabinoid system, in a human being, or in a monogastric animal, or in birds, or in fish,
wherein said composition comprises:
a mixture M comprising or, alternatively, consisting of thymol;
wherein said composition further comprises a controlled release lipid matrix embedding or incorporating said mixture M comprising or, alternatively, consisting of thymol wherein said controlled release lipid matrix comprises or, alternatively, consists of at least one saturated or unsaturated, free or esterified fatty acid having a number of carbon atoms comprised in the range from C10-C30, and/or at least one triglyceride having saturated or unsaturated fatty acid chains, having a number of carbon atoms comprised in the range from C6-C30 and/or at least one wax having a number of carbon atoms comprised in the range from C16-C36;
wherein said lipid matrix allows a gastroprotection and/or a controlled release of the mixture M, preferably of the thymol contained therein, into the bowel;
and, optionally,
at least one acceptable pharmaceutical or food grade additive and/or excipient.

2. The composition for use according to claim 1, wherein said composition is for use in a monogastric mammal, such as in pigs; preferably a monogastric animal or monogastric mammal in the weaning phase, like in weaning pigs.

3. The composition for use according to claim 1, wherein said disease or symptom is selected from: chronic irritable bowel disease (IBD), Crohn's syndrome or disease, ulcerative colitis, indeterminate colitis, microscopic colitis, collagenous colitis, lymphocytic colitis, ischemic colitis, diversion colitis, pouchitis, irritable bowel syndrome (IBS), diarrhoeic IBS, constipation IBS, alternating IBS, unclassified IBS, dyspepsia, nausea, vomiting, constipation, diarrhoea, abdominal bloating, tympanites and physical fatigue.

4. The composition for use according to claim 1, wherein said controlled release lipid matrix comprises or, alternatively, consists of at least one hydrogenated fatty acid of plant origin having a number of carbon atoms comprised in the range from C14-C24 and/or at least one hydrogenated triglyceride of plant origin having a number of carbon atoms comprised in the range from C14-C24 and/or at least one wax of animal origin having a number of carbon atoms comprised in the range from C24-C36; preferably, wherein said fatty acid, triglyceride or wax is selected from: palm oil, sunflower oil, corn oil, rapeseed oil, peanut oil, soybean oil, olive oil, beeswax, and mixtures thereof.

5. The composition for use according to claim 1, wherein said mixture M further comprises at least one further first active ingredient, preferably deriving from a phytocompound (botanicals), selected from group (I) comprising or, alternatively, consisting of carvacrol, eugenol, capsaicin, turmeric, vanillin, cinnamaldehyde, diallyl disulfide, camphor, limonene, rosmarinic acid, p-cymene, γ-terpinene, α-pinene, α-thujone, 1,8-cineole, verbascoside, tannins, menthol, linalool (linalool C10H18O (CAS No. 78-70-6), terpineol (terpineol C10H18O (CAS No. 98-55-5), anthocyanins (anthocyanin), catechins, alpha-zingiberene (C15H24 CAS No. 495-60-3) and mixtures thereof.

6. The composition for use according to claim 1, wherein said composition comprises thymol and carvacrol, or thymol and vanillin, or thymol and menthol, or thymol and linalool (linalool C10H18O (CAS No. 78-70-6), or thymol and terpineol (terpineol C10H18O (CAS No. 98-55-5), or thymol and anthocyanins (anthocyanin), or thymol and catechins, or thymol and alpha-zingiberene (C15H24 CAS No. 495-60-3) and mixtures thereof; or thymol, carvacrol and capsaicin, or thymol, carvacrol and cinnamaldehyde, or thymol, eugenol and verbascoside.

7. The composition for use according to claim 1, wherein besides thymol and, optionally, said further first active ingredient (botanicals) selected from group (I)t, the mixture M contained in said composition comprises an organic acid or a salt thereof with an alkaline or alkaline earth metal cation, wherein said organic acid is selected from group (II) comprising or, alternatively, consisting of lactic acid, malic acid, benzoic acid, fumaric acid, sorbic acid, citric acid, octanoic acid, heptanoic acid, butyric acid, dodecanoic acid and mixture thereof; preferably it may comprise thymol and sorbic acid, or thymol, sorbic acid and citric acid, or thymol and benzoic acid, or thymol, sorbic acid, citric acid and benzoic acid, or thymol, carvacrol and sorbic acid, or thymol, carvacrol, sorbic acid and citric acid, or thymol, carvacrol, cinnamaldehyde and sorbic acid, or thymol, carvacrol, cinnamaldehyde, sorbic acid and citric acid, or thymol and butyric acid, or thymol, citric acid and dodecanoic acid.

8. The composition for use according to claim 1, wherein said mixture M further comprises at least one further second active ingredient selected from group (III) consisting of:
bacterial strains or probiotic bacterial strains belonging to the genus Lactobacillus, Bifidobacterium, Streptococcus, Leuconostoc, Pediococcus, Enterococcus, Saccaromyces; and/or
prebiotics, such as for example inulin, lactulose, lactitol, mannooligosaccharides, fructooligosaccharides and galactooligosaccharides, tributyrin; monobutyrin (monobutyrin C7H1404 CAS No. 557-25-5), monolaurin (monolaurin C15H30O4 CAS No. 27215-38-9) and mixtures thereof and/or
salts of metals, such as zinc and copper; and mixtures thereof.

9. The composition for use according to claim 1, wherein said composition is a feed or feed additive for monogastric animals and/or monogastric animals in the weaning or initial growth phase;
preferably for monogastric mammals, more preferably for pigs.

10. A process for the preparation of the compositions according to claim 1, wherein said process provides for a step in which oil-in-water emulsions and, separately, water-in-oil emulsions are prepared, said emulsions containing the ingredients of group (I) and/or group (II) and/or group (III) which constitute the mixture to obtain a composition in solid form.

11. A method for preventive and/or curative treatment of, an inflammatory and/or functional bowel disease or of a related symptom by modulating receptors and/or enzymes of the endocannabinoid system, in subject selected from a human being, a monogastric animal, a bird, or a fish, comprising administering a composition to the subject, wherein said composition comprises:
a mixture M comprising or, alternatively, consisting of thymol;
wherein said composition further comprises a controlled release lipid matrix embedding or incorporating said mixture M comprising or, alternatively, consisting of thymol wherein said controlled release lipid matrix comprises or, alternatively, consists of at least one saturated or unsaturated, free or esterified fatty acid having a number of carbon atoms comprised in the range from C10-C30, and/or at least one triglyceride having saturated or unsaturated fatty acid chains, having a number of carbon atoms comprised in the range from C6-C30 and/or at least one wax having a number of carbon atoms comprised in the range from C16-C36;
wherein said lipid matrix allows a gastroprotection and/or a controlled release of the mixture M, preferably of the thymol contained therein, into the bowel;
and, optionally,
at least one acceptable pharmaceutical or food grade additive and/or excipient.

12. The method of claim 11, wherein said subject is a monogastric mammal, such as in pigs; preferably a monogastric animal or monogastric mammal in the weaning phase, like in weaning pigs.

13. The method of claim 11, wherein said disease or symptom is selected from: chronic irritable bowel disease (IBD), Crohn's syndrome or disease, ulcerative colitis, indeterminate colitis, microscopic colitis, collagenous colitis, lymphocytic colitis, ischemic colitis, diversion colitis, pouchitis, irritable bowel syndrome (IBS), diarrhoeic IBS, constipation IBS, alternating IBS, unclassified IBS, dyspepsia, nausea, vomiting, constipation, diarrhoea, abdominal bloating, tympanites and physical fatigue.

14. The method of claim 11, wherein said controlled release lipid matrix comprises or, alternatively, consists of at least one hydrogenated fatty acid of plant origin having a number of carbon atoms comprised in the range from C14-C24 and/or at least one hydrogenated triglyceride of plant origin having a number of carbon atoms comprised in the range from C14-C24 and/or at least one wax of animal origin having a number of carbon atoms comprised in the range from C24-C36; preferably, wherein said fatty acid, triglyceride or wax is selected from: palm oil, sunflower oil, corn oil, rapeseed oil, peanut oil, soybean oil, olive oil, beeswax, and mixtures thereof.

15. The method of claim 11, wherein said mixture M further comprises at least one further first active ingredient, preferably deriving from a phytocompound (botanicals), selected from group (I) comprising or, alternatively, consisting of carvacrol, eugenol, capsaicin, turmeric, vanillin, cinnamaldehyde, diallyl disulfide, camphor, limonene, rosmarinic acid, p-cymene, γ-terpinene, α-pinene, α-thujone, 1,8-cineole, verbascoside, tannins, menthol, linalool (linalool C10H18O (CAS No. 78-70-6), terpineol (terpineol C10H18O (CAS No. 98-55-5), anthocyanins (anthocyanin), catechins, alpha-zingiberene (C15H24 CAS No. 495-60-3) and mixtures thereof.

16. The method of claim 11, wherein said composition comprises thymol and carvacrol, or thymol and vanillin, or thymol and menthol, or thymol and linalool (linalool C10H18O (CAS No. 78-70-6), or thymol and terpineol (terpineol C10H18O (CAS No. 98-55-5), or thymol and anthocyanins (anthocyanin), or thymol and catechins, or thymol and alpha-zingiberene (C15H24 CAS No. 495-60-3) and mixtures thereof; or thymol, carvacrol and capsaicin, or thymol, carvacrol and cinnamaldehyde, or thymol, eugenol and verbascoside.

17. The method of claim 11, wherein besides thymol and, optionally, said further first active ingredient (botanicals) selected from group (I)t, the mixture M contained in said composition comprises an organic acid or a salt thereof with an alkaline or alkaline earth metal cation, wherein said organic acid is selected from group (II) comprising or, alternatively, consisting of lactic acid, malic acid, benzoic acid, fumaric acid, sorbic acid, citric acid, octanoic acid, heptanoic acid, butyric acid, dodecanoic acid and mixture thereof; preferably it may comprise thymol and sorbic acid, or thymol, sorbic acid and citric acid, or thymol and benzoic acid, or thymol, sorbic acid, citric acid and benzoic acid, or thymol, carvacrol and sorbic acid, or thymol, carvacrol, sorbic acid and citric acid, or thymol, carvacrol, cinnamaldehyde and sorbic acid, or thymol, carvacrol, cinnamaldehyde, sorbic acid and citric acid, or thymol and butyric acid, or thymol, citric acid and dodecanoic acid.

18. The method of claim 11, wherein said mixture M further comprises at least one further second active ingredient selected from group (III) consisting of:
bacterial strains or probiotic bacterial strains belonging to the genus Lactobacillus, Bifidobacterium, Streptococcus, Leuconostoc, Pediococcus, Enterococcus, Saccaromyces; and/or
prebiotics, such as for example inulin, lactulose, lactitol, mannooligosaccharides, fructooligosaccharides and galactooligosaccharides, tributyrin; monobutyrin (monobutyrin C7H1404 CAS No. 557-25-5), monolaurin (monolaurin C15H30O4 CAS No. 27215-38-9) and mixtures thereof and/or
salts of metals, such as zinc and copper; and mixtures thereof.

19. The method of claim 11, wherein said composition is a feed or feed additive for monogastric animals and/or monogastric animals in the weaning or initial growth phase;
preferably for monogastric mammals, more preferably for pigs.
